(19) United States
(12) Patent Application Publication
(10) Pub. No.: US 2013/0317226 A1
(43) Pub. Date: Nov. 28, 2013

(54) INHIBITORS OF THE INTERACTION OF THE SIGMA-1 RECEPTOR WITH hERG FOR USE IN THE TREATMENT OF CANCER

(71) Applicants: Olivier Soriani, Nice (FR); Mauro Franck Borgese, Saint-Laurent-du-Var (FR); Sonia Martial, Nice (FR)

(72) Inventors: Olivier Soriani, Nice (FR); Mauro Franck Borgese, Saint-Laurent-du-Var (FR); Sonia Martial, Nice (FR)

(73) Assignees: UNIVERSITE NICE SOPHIA ANTIPOLIS, Nice Cedex (FR); CENTRE NATIONAL DE LA RECHERCHE SCIENTIFIQUE - CNRS, Paris Cedex 16 (FR)

(21) Appl. No.: 13/982,775

(22) PCT Filed: Jan. 31, 2012
(86) PCT No.: PCT/FR2012/050213

§ 371 Date: Jul. 31, 2013

(30) Foreign Application Priority Data
Jan. 31, 2011 (FR) 1150709

(51) Int. Cl. A61K 31/439; A61K 31/137

(52) U.S. Cl. CPC A61K 31/439; A61K 31/137
USPC 546/97; 564/373

(57) ABSTRACT

The present invention relates to the use of the Sigma-1 receptor (Sig1R) in the context of the post-transcriptional regulation of the membrane expression of ion channels.

The present invention can be used in the field of the treatment of diseases involving ion channels. These are, for example, nervous system diseases, neurodegenerative diseases, heart diseases, and cancer.

### TECHNICAL FIELD

The present invention relates to the use of the Sigma-1 receptor (Sig1 R) for regulating ion channel expression at the post-transcriptional level.

Thus, Sig1R silencing in cells induces a reduction in the density of current generated by ion channels, correlated with a reduction in the Sig1R expression level, and also a decrease in specific adhesion to fibronectin (FN). A contrario, the coexpression of hERG and Sig1R causes a potentiation of the current and of the level of protein expressed.

The present invention can be used in the manufacture of a medicament intended for the treatment of diseases involving ion channels. These are, for example, nervous system diseases, such as, for example, epilepsy, neurodegenerative diseases such as, for example, Alzheimer's disease, heart diseases, and cancer.

In the description below, the references between square brackets ([ ]) refer to the list of references provided at the end of the text.

### PRIOR ART

Ion channels are proteins inserted into the membranes of the cells of all living beings. These channels allow ions (potassium, sodium, calcium and chlorine) to cross the membrane. This passage creates an electric current, the functions of which are fundamental: nerve messages, muscle and heart contraction, hormone secretion, body fluid composition. Only recently, these channels have also been implicated in tumour proliferation and metastatic aggressiveness. Indeed, the aberrant expression of channels of different nature has been observed in numerous primary cancers in humans, and is frequently correlated with the aggressiveness of the tumour. As a general rule, ion channels exert pleiotropic effects on the physiology of neoplastic cells. For example, by regulating the membrane potential, the channels control intracellular Ca²⁺ flux and, consequently, the cell cycle. Their effects on mitosis can also depend on the regulation of cell volume, most commonly in cooperation with chloride channels. Ion channels are also involved in the final neoplastic steps, the stimulation of angiogenesis, cell/extracellular matrix interaction or the regulation of cell motility. Thus, the contribution of ion channels in the neoplastic phenotype extends from the control of cell proliferation and of apoptosis, to the regulation of invasion and of metastatic propagation. However, while their involvement is increasingly demonstrated, the regulation and the molecular mechanisms associated with ion channels in a tumour context remains to be specified. There are multiple mechanisms governing the effects of ion channels and they involve a cascade of pathways of intracellular signalling generally triggered after the formation of protein complexes with other membrane proteins, such as integrins or growth factor receptors. Finally, in a tumour context, ion channels are generally diverted from their primary function in order to participate in the tumour phenotype. Thus, better knowledge of their expression profile, of their partners, and of the structure/function relationship of channel complexes would potentially open the way to new targeted therapeutic strategies. Indeed, some chemical molecules are capable of directly blocking these channels and of slowing the growth of cancerous tumours in vitro. However, the use of these molecules in patients is dangerous since the channels of tumours are the same as those found in healthy tissue (muscles or the brain, etc.).

There is therefore a real need for pharmacological compounds which overcome the faults, drawbacks and obstacles of the prior art, in particular for compounds capable of targeting ion channels in cancers without impairing the functions of healthy organs, of reducing costs, and of thus improving anticancer therapies.

The Sigma-1 receptor (Sig1R) is a ubiquitous protein anchored in the cell membrane. This protein is overexpressed in tumour cells to the point of being proposed as a biological tumour marker in cell imaging [1] and implicated in cell proliferation [2]. Its most well-described aspect is the modulation of ion channels, although its mode of action remains unknown. The activation of Sig1R by specific exogenous ligands changes the electrical properties of the membrane, which is a consequence of the modification of the activation/inactivation kinetics of chloride channels and voltage-dependent potassium channels (M, I_{A}, KV1.5) [5-7]. Recently, it has been shown that Sig1R controls various families of ion channels, including voltage-dependent potassium channels (Kv1.3) and VRCC (Volume Regulated Chloride Channel) channels. Furthermore, Sig1R-Kv1.3 or Sig1R-VRCC interactions participate in several cell events: the cell cycle, resistance to apoptosis [8,9], but not in interaction with the extracellular matrix and the formation of metastases, as is the case, for example, for the ERG channel (ether-a-go-go-related channel). Furthermore, the channels modulated by Sig1R in these studies are expressed both in the healthy tissue and in the tumour tissue, whereas hERG is absent from the healthy tissue and therefore presents itself as a marker for numerous tumours.

The hERG channel is a cardiac voltage-dependent K⁺ channel, the essential function of which is to regulate the time which separates two successive beats, and the aberrant expression of which has been characterized in numerous cancers, including breast cancers, colon cancers, neuroblastomas and myeloid leukaemias [10]. The expression of hERG is correlated with the invasive potential of the cells, and the study of the mechanisms involved demonstrates an atypical signalling pathway: the stimulation of integrins by the extracellular matrix (ECM) activates hERG, this being a step required for the recruitment of Rac1 and FAKs and association with the VEGF receptor FLT-1. The channel macrocomplex thus formed potentiates migration, invasion and angiogenesis [10].

Consequently, there is currently no therapeutic tool specifically targeting these channel macrocomplexes of Sig1R/ion channel which make it possible to inhibit cell proliferation, while limiting the side effects resulting from the use of molecules having a non-targeted action.

### Description of the Invention

The inventors have now demonstrated, entirely unexpectedly, that it is possible to regulate ion channels at the post-transcriptional level, for example the hERG (human Ether-a-go-go-Related Gene) ion channel, via the Sigma-1 receptor (Sig1R), in a chronic myeloid leukaemia cell model: K562 cells which express a single type of integrin, α5β1, which is the fibronectin (FN) receptor, and the hERG channel. An analogous result was obtained in another cell line: MDA-MD-435s breast cancer cells. This regulation is carried out through a functional interaction between the two proteins. Thus, Sig1R makes it possible to modulate the maturation and the membrane stability of hERG without modifying the kinetic properties of said channel.

In addition, the inventors have demonstrated that the sigma-1 receptor is an integral part of the signalling pathways which regulate the adhesion of cells to the extracellular matrix. Indeed, Sig1R silencing in K562 cells causes a decrease in specific adhesion to fibronectin (FN). The consequences of these observations are that, by targeting Sig1R, it is possible to act on the tumour formation programme by interfering in the cell signalling pathways assigned to ion channels.

Likewise, this mechanism of regulation can be transposed to the nervous or cardiac system, where Sig1R is present, in order to allow the re-examination of certain pathological conditions involving ion channels, outside any tumour context.

The subject of the present invention is therefore the use of a modulator of channel macrocomplexes comprising or consisting of Sig1R and of ion channels for obtaining a medicament intended for the post-transcriptional regulation of the membrane expression of ion channels for the treatment of diseases involving said ion channels.

The subject of the present invention is therefore a ligand which modulates the interaction between Sig1R and an ion channel, for use thereof as a medicament intended for the post-transcriptional regulation of the membrane expression of said ion channel; that is to say for the treatment of diseases involving said ion channels.

The subject of the invention is therefore a ligand which modulates the number of ion channels associated with the membrane, for use thereof as a medicament intended for the post-transcriptional regulation of the membrane expression of said ion channel; that is to say the treatment of diseases involving said ion channels.

For the purpose of the present invention, the expression “ligand which modulates” is intended to mean any molecule capable of modulating the activity and/or the expression of Sig1R and/or of the ion channel. For example, it is a Sig1R ligand capable of modulating the maturation and the membrane stability of said ion channel without modifying the kinetic properties thereof.

For the purpose of the present invention, the term “medicament” is intended to mean any tool or any therapeutic means known to those skilled in the art (for example, substance or composition) presented as having curative and/or preventive properties with regard to human or animal diseases, and also any product which can be administered to humans or to animals, for the purpose of establishing a medical diagnosis and/or restoring, correcting and/or modifying their organic functions.

For the purpose of the present invention, the expression “diseases involving ion channels” is intended to mean nervous system diseases such as, for example, epilepsy, neurodegenerative diseases such as, for example, Alzheimer's disease, heart diseases such as, for example, long QT syndrome, and cancer.

According to one particular embodiment of the invention, said medicament is intended for the treatment of cancer.

According to one particular embodiment of the invention, said medicament inhibits the membrane expression of ion channels, preferably of potassium (K⁺) ion channels, preferentially of channels belonging to the “ether-a-go-go-related gene” (ERG) family.

Other advantages may become further apparent to those skilled in the art on reading the examples below, illustrated by the appended figures, given by way of illustration.

### BRIEF DESCRIPTION OF THE FIGURES

FIG. 1 represents the characterization of the hERG currents in K562 cells. (A), tail currents recorded at −120 mV following a prepulse at +40 mV (Epp) in order to fully activate the hERG channels, in the absence (control) or presence of E-4031 (1 μM). (B), detail of the tail currents presented in (A). (C), families of tail currents recorded following prepulses of −70 to 40 mV, in the absence (control) or presence of E-4031 (1 μM). The bottom graph represents the graphic subtraction of the previous two graphs (control and (+) E-4031). (D), IN (current/voltage) curves corresponding to the traces shown in C. The tail current amplitudes are plotted as a function of the prepulse potential (Epp).

FIG. 2 represents the reduction in the hERG current by the sigma ligands in the K562 cells. (A), change over time of the hERG current recorded at −120 mV after a prepulse of 40 mV in two distinct cells. Igmesine (Igm, left, 10 μM) or (+)pentazocine ((+)Ptz, right, 10 μM) applied in the extracellular medium for the period of time represented by the black bar. (B), families of hERG currents recorded at −120 mV after prepulses of −70 to 40 mV in a single cell in the absence (left, control) or presence of igmesine (10 μM, right, Igm). (C), corresponding IN curves. The tail current amplitudes are represented as a function of the prepulse potential (Epp). (D), curve of deactivation of the hERG current at −120 mV in the absence (white columns) or presence of igmesine (10 μM, black columns). The deactivation curve was adjusted using an exponential double function. The values represent the mean±standard error. Comparison of the means carried out using the Student's t test.

FIG. 3 represents Sig1R silencing in K562 and MDA-MB-435 cells. (A), Western blots of K562 cells transduced with a random shRNA (shRD) or an shRNA directed against Sig1R (shSigl R), and visualized with anti-actin (upper line) or anti-Sig1R (lower line) antibodies. (B), Western blots of MDA-MB-435 cells transduced with a random shRNA (shRD) or an shRNA directed against Sig1R (shSig1R), and visualized with anti-actin (upper line) or anti-Sig1R (lower line) antibodies.

FIG. 4 represents the reduction in the hERG current density in K562 cells via Sig1R silencing. (A), families of hERG currents recorded in response to the protocol described in 1C. The traces were obtained from K562 cells expressing a random shRNA (K562 shRD, upper) or an shRNA directed against Sig1R (K562 shSigl R, lower). (B), mean of the IN curves corresponding to the traces of the K562 shRD cells (black squares) and K562 shSigl R cells (black circles). (C), histogram showing the mean of the current amplitudes in the K562 shRD and K562 shSigl R cells. (D), curve of activations deduced from C. (E), speed of deactivation of the hERG current at −120 mV in the K562 shRD cells (white columns) and K562 shSigl R cells (black columns). The deactivation curve was adjusted using an exponential double function. The values represent the mean±standard error, Student's t test.

FIG. 5 represents the impairment of the hERG protein expression in K562 cells via Sig1R silencing. (A), amount of mRNA of the hERG gene expressed in these cells transduced with a non-targeted shRNA (shRD) and transduced with an anti-Sig1R shRNA (shSig1R). NS means not significant (Student's t test). (B), Western blots visualized with anti-hERG (upper block) or anti-tubulin (lower block) antibodies, carried out in the K562 shRD and K562 shSig1R cells. (C), histogram of the densitometric analysis of the mature and immature isoforms of hERG in the K562 shSigl R cells (black columns) compared with the K562 shRD cells (white columns). The values correspond to the hERG/tubulin densitometric ratio. (D), efficiency of the intracellular trafficking of hERG1b in the K562 shRD and shSig1R cells (black columns). The trafficking efficiency is calculated as the (mature)/(mature+immature) densitometric ratio. *p<0.05 (Student's t test).

FIG. 6 represents the stimulation of the hERG currents in Xenopus oocytes via the expression of Sig1R. (A), family of tail currents in non-injected oocytes (NI), injected with the hERG cRNA, and injected with the hERG and Sig1R cRNAs. The voltage protocol is described in 1C. (B), Relative current amplitudes at −120 mV in non-injected oocytes (NI), oocytes injected with the hERG RNA, and oocytes injected with the hERG and Sig1R cRNAs (arbitrary units, 1 corresponding to the mean current recorded in the oocytes injected with the hERG cRNA). The values represent the mean±standard error of n independent experiments, as indicated in the graph. (C), curves of activation of the hERG channels recorded in oocytes injected with the hERG cRNA (black squares) and injected with the hERG and Sig1R cRNAs (black circles). The values represent the mean±standard error of 6 independent experiments (n=6). (D), curves of inactivation of the hERG channels recorded in oocytes injected with the hERG cRNA (black squares) and injected with the hERG and Sig1R cRNAs (black circles). The values represent the mean±standard error of 8 independent experiments (n=8).

FIG. 7 represents the modulation of the expression of hERG by Sig1R via a direct interaction. (A), Western blots visualized with an anti-Sig1R antibody in: non-injected oocytes (NI), oocytes injected with the hERG1a cRNA, and oocytes injected with the hERG1a and Sig1R cRNAs (upper block) and in: non-injected oocytes (NI), oocytes injected with the hERG1b cRNA, and oocytes injected with the hERG1b and Sig1R cRNAs (lower block). The hERG1a, hERG1b and Sig1R cRNAs were injected at the concentrations of 25 pg, 75 pg and 5 ng/oocyte, respectively. (B), an oocyte lysate protein immunoprecipitation was carried out with anti-Myc antibodies in non-injected oocytes (NI), oocytes injected with hERG1a RNA, and oocytes injected with the hERG1a and Sig1R RNAs. The immunocomplexes were visualized with anti-hERG (upper panel) or anti-Sig1R (lower panel) antibodies. The hERG1a and Sig1R cRNAs were injected at a concentration of 15 ng/oocyte.

FIG. 8 represents the inhibition of the adhesion of K562 cells to FN by igmesine, E-4031 and Sig1R silencing. (A), histogram representing the percentage adhesion to fibronectin (FN) of K562 cells treated with E-4031 (10 μM), E4031+igmesine (10 μM each) or igmesine alone (10 μM), compared with the “control” conditions (ctl, 100%). The values represent the mean±standard error of 6 to 9 independent experiments. NS: not significant (Student's t test). (B), percentage adhesion to FN of the K562 shRD (white columns) and K562 shSig1R (black columns) cells. The values represent the mean±standard error of 12 independent experiments. ***p<0.001 (Student's t test). In one experiment, each value is the mean of three distinct wells.

FIG. 9 represents the expression of hERG at the surface of the plasma membrane, visualized by flow cytometry in the shRD and shSigl R cells (anti-h ERG antibody directed against an extracellular loop of the channel). Right-hand panel: corresponding histogram. *p<0.02, Mann-Whitney.

FIG. 10 represents the improvement in the maturation and the stability of hERG by Sig1R (A), cells transduced with hERG+cmyc-GFP (control group) or hERG+cmycSig1R, incubated for 10 min with 35S methionine, and washed in medium without radioactivity. At various times, HEK 2963 cell lysates were prepared and the hERG protein was immunoprecipitated. Its maturation profile was analyzed by Western blot. (B), cells incubated for 1 hour and analyzed over a period of 8 hours. hERG was immunoprecipitated and the mature form was visualized by Western blot.

FIG. 11 represents the invasive capacity of “control” K562 shRD cells (left-hand panel) and “shSig1R” Sig1R-knockout K562 cells (right-hand panel) using injections into the yolk sac of zebrafish embryos. The cells were labelled red with a fluorescent label (CM-DIL, cf. arrows and arrowheads). The histogram represents the percentage of embryos with more than 3 cells inside the yolk sac after 48 hours.

### EXAMPLES

### Example 1

### The Sigma Ligands Inhibit the hERG Current in K562 Cells

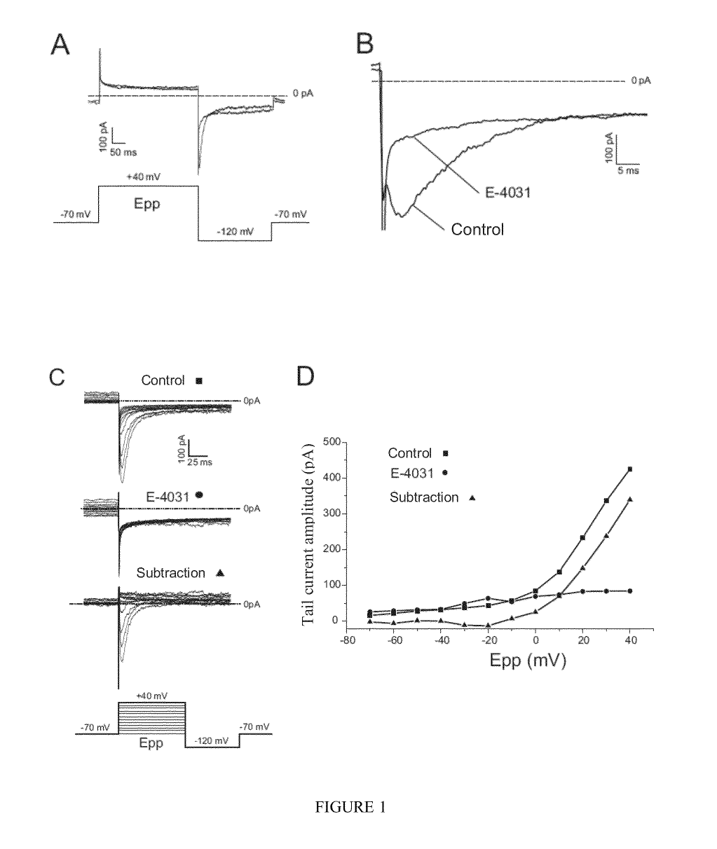

“Patch-clamp” electrophysiology experiments were carried out in the whole cell configuration. The extracellular saline solution bathing the cells contains a high potassium concentration in order to improve the amplitude of the inward potassium current at −120 mV. The hERG currents were analyzed as abovementioned tail currents at −120 mV following prepulses of −70 to 40 mV. This protocol makes it possible to record transient inward currents, the amplitude of which correlates with the depolarization involved during the prepulses. These currents were completely eliminated by the perfusion of the hERG-specific inhibitor E-4031 (1 μM) [11] (FIG. 1 A,B). In addition, the graphic subtraction revealed that E-4031 inhibits a voltage-dependent conductance (FIG. 1 C, D). In conclusion, these data confirmed the presence of functional hERG channels in the K562 cell line [12].

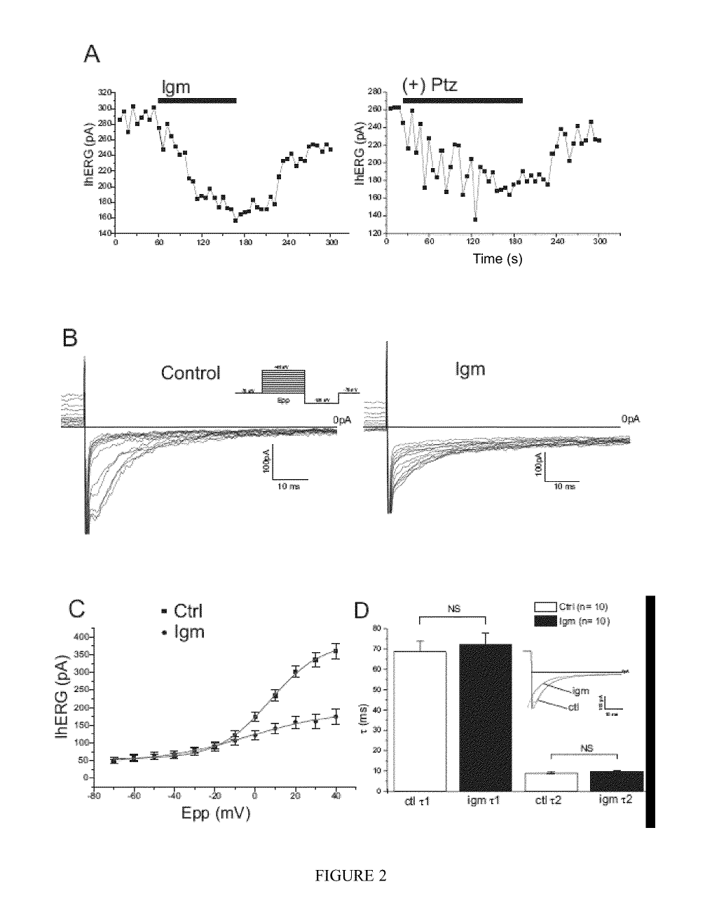

In order to verify a potential interaction between Sig1R and hERG, the effects of selective Sig1R ligands, i.e. igmesine and (+)pentazocine [7, 9, 13, 14] were analyzed with respect to the current. The extracellular applications of igmesine or of (+)pentazocine reversibly inhibited the currents. The current was reduced by 40.85±2.83% (n=10) and 21.19±1.77% (n=3) for igmesine and (+)pentazocine, respectively (10 μM each). Maximum inhibition occurred within 3 minutes following the beginning of the drug applications (FIG. 2A). The effects of igmesine (10 μM) were then studied on IN curves recorded at from −70 to 40 mV. Igmesine (10 μM) caused a spectacular reduction in the amplitude of the maximum current (FIG. 2 B, C) suggesting that the drug mainly affected the current density. Nevertheless, processing of the curves according to a Boltzmann function revealed an apparent 10 mV shift towards the left of the half-activation voltage value (FIG. 2B, table 1).

TABLE 1

| | V_{1/2} | slope |
|---|---|---|
| K562 | 7.1 ± 2.3 mV | 17.4 ± 3.6 |
| n = 6 | | |
| K562 = igmesine | −4.1 ± 2.9 mV | 23.1 ± 2.7 |
| (10 μM) | p < 0.009 | N.S. |
| n = 6 | (T test) | (T test) |
| shRD K562 | 23.4 ± 3.1 mV | 12.2 ± 0.8 |
| n = 7 | | |
| shSig1R K562 | 22.8 ± 2.7 mV | 17 ± 2.5 |
| n = 4 | N.S. | N.S. |
| | (T test) | (T test) |

However, igmesine changed neither the fast nor the slow deactivation components of the hERG tail current recorded at −120 mV (FIG. 2D). These results indicated for the first time that Sig1R modulates hERG channels. This hERG current inhibition by the Sigma receptor agonists is not due to a modification of the electrical characteristics of the channels, but is the result of a decrease in the number of active channels at the cell membrane.

### Example 2

### Sig1R Silencing Decreases the hERG Current Density in K562 Cells

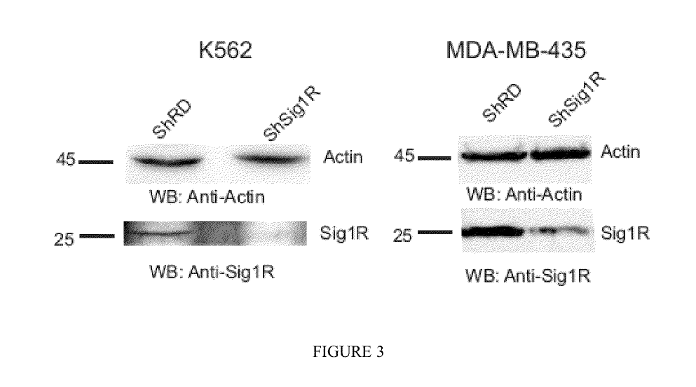

The effects of Sig1R silencing were studied on the hERG activity of K562 cells. The K562 cells were transduced with a retrovirus containing either a random shRNA (short hairpin RNA) or an shRNA directed against Sig1R, giving rise to two cell populations called, respectively, shRD and shSig1R. The Western blot experiments revealed a spectacular decrease in Sig1R expression in the shSigl R cell line (FIG. 3, left). The same result was obtained in MDA-MB-435s cells expressing the same shRD and shSigl R (FIG. 3, right) demonstrating the effectiveness and the specificity of the Sig1R-directed shRNA used here.

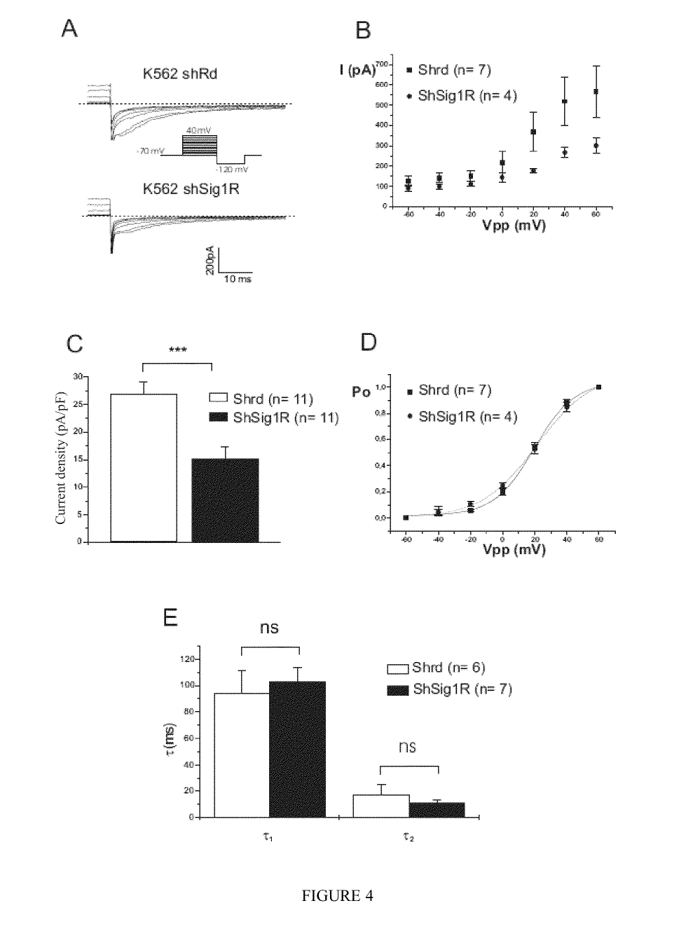

Patch-clamp experiments were then carried out in the K562 shRD and shSigl R cell lines in order to analyze the possible consequences of Sig1R silencing on the properties of hERG. Interestingly, the tail current families recorded at −120 mV in shSig1R were clearly smaller in amplitude than those in the shRD cells (FIG. 4A). This decrease in the current amplitude was observed for activation potentials of between −20 and +60 mV (FIG. 4B). At +40 mV, the current density was reduced by 44% (FIG. 4C). It is noted that Sig1R silencing did not shift the activation curve (FIG. 4D, table 1). In the same way, no significant difference could be observed for fast and slow deactivation time constants in the two cell populations (FIG. 4E). In conclusion, these data suggested that Sig1R silencing reduces the current density without impairing the kinetic parameters of hERG in K562 cells. Here again, the decrease is a reflection of a decrease in the number of active channels at the cell membrane.

### Example 3

### Sig1R Silencing Modulates the Expression of hERG in K562 Cells

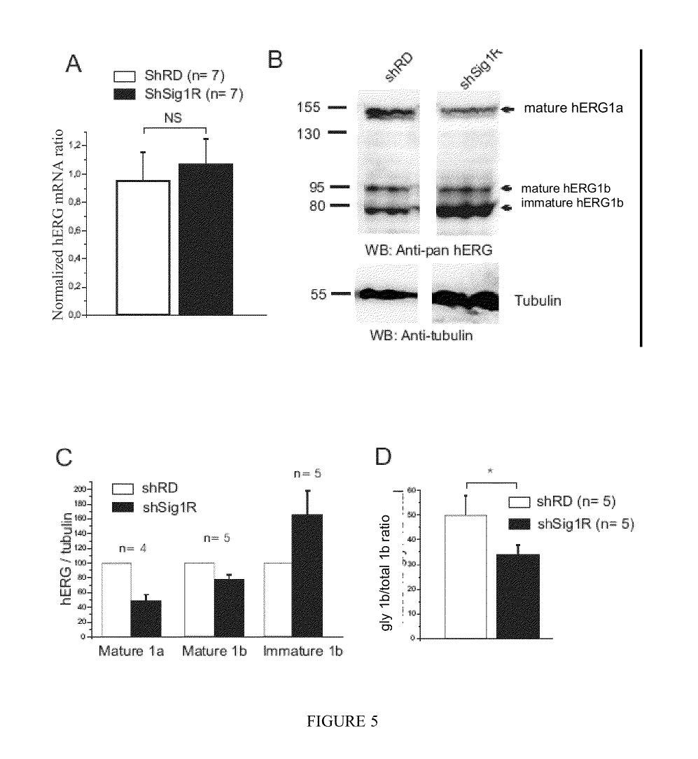

In order to understand more clearly the link between the expression of Sig1R and hERG currents, the expression of hERG was analyzed in the two cell lines, using real-time PCR and Western blot analysis. The hERG mRNA levels were not significantly different in the shRD and shSigl R cell lines, excluding any Sig1R-dependent modulation of hERG transcription which could explain the decrease observed in the current density (FIG. 5A). At the protein level, the Western blots using an anti-pan-hERG antibody revealed that K562 cells expressed two different isoforms of hERG, i.e. hERG1a, corresponding to the complete protein and the splice variant hERG1b which has a truncated N-terminal end [15]. The band at 155 kDa representing the fully glycosylated form (mature hERG1a) was regularly detected, whereas the 135 kDA immature form (partially glycosylated) of hERG1a was rarely visible (FIG. 5B, left). However, in some experiments, the 135 kDa immature form could clearly be observed (not represented). On the other hand, the spliced isoform hERG1b constantly appeared in the form of two distinct bands, the fully glycosylated mature form of 95 kDa and the immature form, at 80 kDa (FIG. 5B, left). The two mature glycoforms of hERG are known to represent the channel subunit fraction which is located at the plasma membrane after having left the endoplasmic reticulum (ER) so as to be matured through the Golgi apparatus and to reach the cell surface. Interestingly, the Sig1R silencing modified the hERG expression profile: in the shSigl R cells, a decrease in the amount of the two mature forms hERG1a and hERG1b could be observed. This was accompanied by an increase in the hERG1b immature form (FIG. 5 B,C). In the experiments for which the hERG1a immature form of 135 kDa was clearly detected, its expression level was also increased in the shSig1R cells compared with the shRD cells (not represented). The quantification of the mature hERG1b/total hERG1b ratio demonstrated that the Sig1R silencing significantly reduced the maturation of hERG1b, [16] (FIG. 5A). In conclusion, these results demonstrated for the first time that Sig1R modifies the post-transcriptional regulation of the hERG channel. Sig1R silencing induces a decrease in the mature form of hERG1a (150 kDa) and also an accumulation of the immature form of the hERG1b splice variant (80 kDa), indicating a decrease in efficiency of trafficking of the protein. These expression profiles are entirely in agreement with the decrease in the current observed.

### Example 4

### The Expression of Sig1R Potentiates the hERG Current Density in Xenopus oocytes

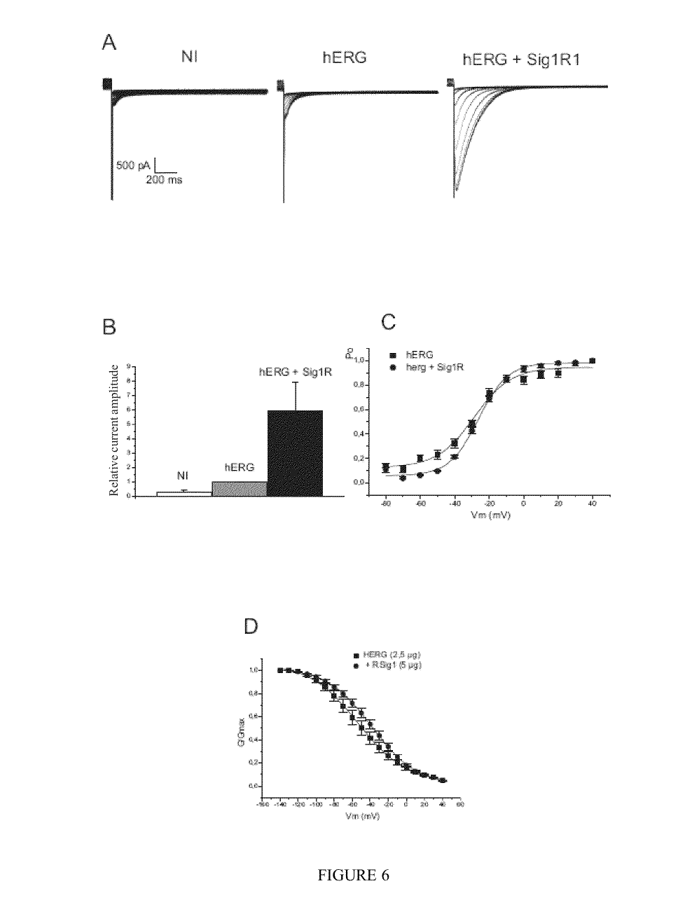

In order to confirm the atypical function of Sig1R on the expression of hERG revealed here using K562 cells, the experiments were carried out in Xenopus oocytes. The injection of hERG cRNA (25 pg/oocyte) into oocytes induced the appearance of currents that were absent from the oocytes injected with water (FIG. 6A, left and middle). The co-injection of Sig1R cRNA (5 ng/oocyte) with the same low concentration of hERG cRNA multiplied the current amplitude five-fold compared with the eggs expressing hERG alone (FIG. 6 A,B). The potentiation of the current depends on the concentrations of Sig1R cRNA injected, but disappears for high concentrations of hERG cRNA 15 ng/oocyte, not represented). The expression of Sig1R did not affect the activation parameters (FIG. 6C, table 2). A shift to the right of the half-inactivation potential value can be noted, but this difference is not significant (FIG. 6D and table 2).

TABLE 2

| | Activation | | Inactivation | |
|---|---|---|---|---|
| | V_{1/2} | slope | V_{1/2} | slope |
| hERG1a | −28.7 ± 0.3 mV | 10.2 ± 2.7 | −54.1 ± 7.3 mV | −22.8 ± 1.3 |
| n = 6 | | | | |
| hERG1a + Sig1R | −26.8 ± 1.0 mV | 9.2 ± 0.44 | −38.7 ± 4.1 mV | −24.1 ± 1.9 |
| n = 6 | N.S. | N.S. | N.S. | N.S. |
| | (T test) | (T test) | (T test) | (T test) |

These data indicate that the expression of Sig1R regulates mainly the hERG current density.

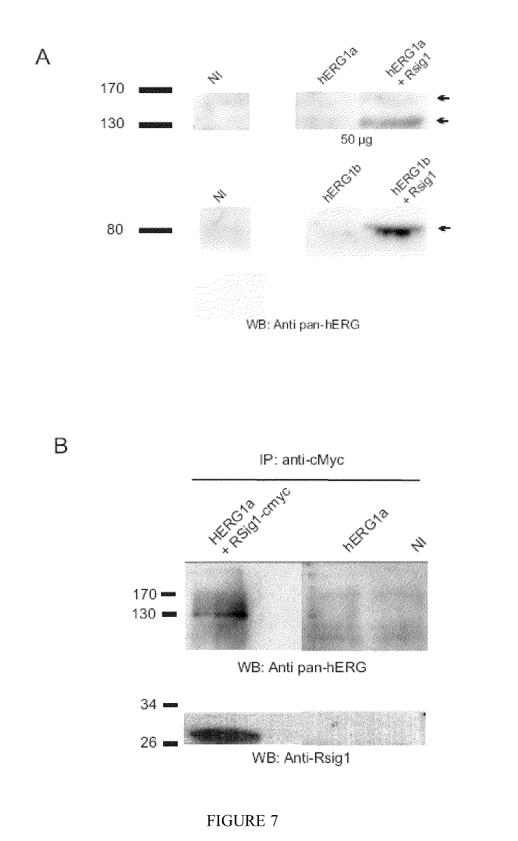

In order to confirm these data, Western blot analyses of the membrane proteins of injected and control oocytes were carried out. The results show that, for low concentrations of hERG1a cRNA injected alone, faint bands corresponding to the immature and mature hERG1a proteins were detected; the co-injection of hERG1a and Sig1R cRNA resulted in a clear increase in the two hERG1a proteins (FIG. 7A, top). In the Western blot experiments carried out with the hERG1b mRNA, only the immature band of 80 kDa could be clearly detected, which is in agreement with the previous results demonstrating that the hERG1b trafficking efficiency is down-modulated in the absence of hERG1a subunits [17]. Once again, the co-injection of hERG1b with the Sig1R cRNA caused a clear increase in the intensity of the 80 kDa band (FIG. 7A, bottom). On the other hand, the expression of Sig1R did not increase the hERG protein level when high concentrations of Sig1R cRNA 15 ng/oocyte) were injected (not represented). These results showed that the expression of Sig1R stimulates the hERG current via an increase in the amount of hERG channel. Thus, these experiments conclude that Sig1R stabilizes the channels at the plasma membrane.

The existence of a direct interaction between hERG and Sig1R was explored in Xenopus oocytes via the injection of cRNA of hERG1a or of hERG1a+cMyc-Sig1R. It should be noted that it has been demonstrated that Sig1R tagged with cMyc in the NH₂-terminal portion is functionally expressed in HEK293 cells [9] and increases the hERG current in Xenopus oocytes in the same way as native Sig1R cRNA does (not represented). The Sig1R receptors were immunoprecipitated using an antibody directed against the cMyc tag and the result of the immunoprecipitation was resolved on an SDS PAGE gel. The Western blot was visualized using the anti-pan-hERG antibody and the anti-Sig1R antibody. As shown in FIG. 7B, in the oocytes co-injected (Sig1R-cMyc+hERG1a), the two proteins co-immunoprecipitate, thus demonstrating a direct functional interaction between Sig1R and hERG.

### Example 5

### Sig1R Increases the Maturation and the Stabilization of hERG at the Cell Surface [Crottès et al., J. Biol. Chem., 286(32): 27947-27958, 2011] [19]

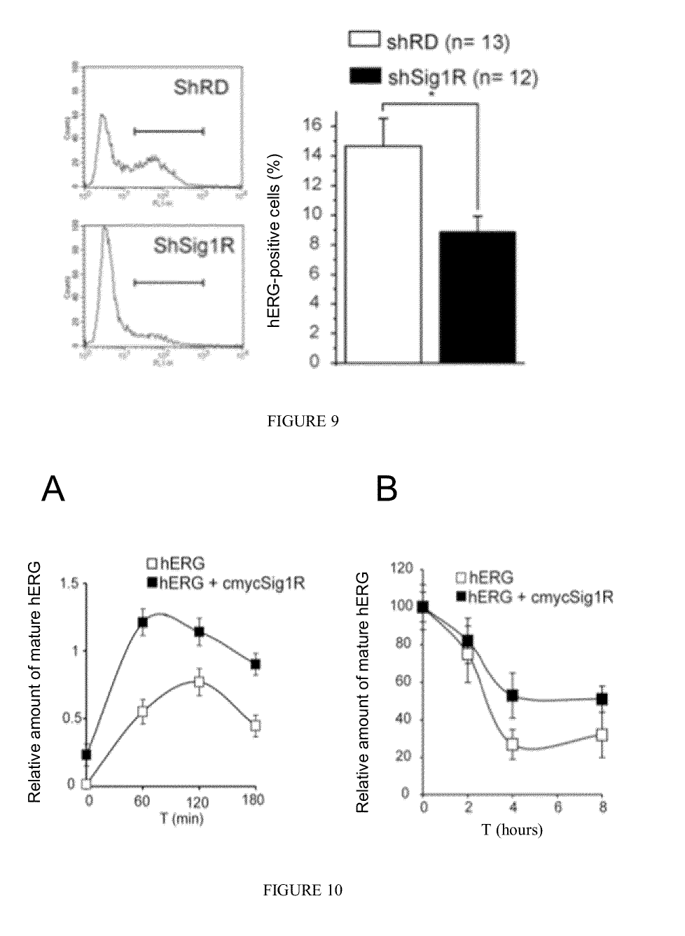

The flow cytometry made it possible to confirm that Sig1R indeed increases the amount of hERG channels at the surface of the shRD cells compared with the shSig1R cells. The labelling was carried out using an anti-hERG antibody directed against an extracellular loop of the channel. The results showed that the Sig1R silencing (shSig1R) reduced by 35% the expression of hERG at the cell surface (FIG. 9).

The study of the rate of maturation of hERG was also carried out by performing “pulse-chase” studies on HEK cells expressing cmyc-Sig1R. The results showed that the overexpression of Sig1R increased the rate of maturation of hERG (FIG. 10A) and also its stability at the plasma membrane (FIG. 10B).

### Example 6

### Sig1R Modulates the Adhesion of K562 Cells to FN

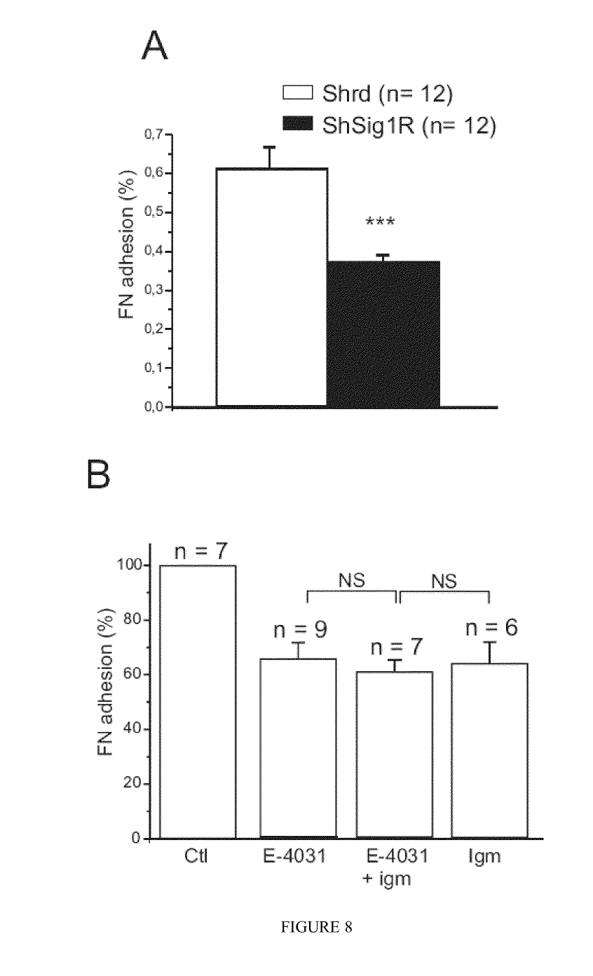

The functional association between hERG channels and integrins has recently been demonstrated [18,10]. The role of the Sig1R/hERG interaction on integrin-dependent cell adhesion to the extracellular matrix (ECM) has been tested in vitro. The expression of a single integrin subtype, i.e. integrin α₅β₁, has been reported in K562 cells [18]. This integrin has a high affinity for fibronectin (FN), a component of the extracellular matrix (ECM). Cell adhesion to ECM-coated wells was significantly inhibited in the shSig1R cells compared with the shRD cells (≈40%, FIG. 8A). In the wild-type cells, the hERG channel blocker (E-4031) and igmesine inhibited FN-dependent specific cell adhesion. Interestingly, igmesine did not produce an additive effect compared with the K562 cells treated with E-4031 (FIG. 8B). Likewise, the Sigma ligands no longer have any effect on the adhesion of the shSig1R cells (not represented). These results indicate that Sig1R inhibits the specific cell adhesion to FN via interaction with the hERG/integrin α₅β₁ complex as previously described in myeloid leukaemia cells [10].

### Example 7

### Role of Sig1R in Invasive Potential In Vivo

The zebrafish is a model that is increasingly used in cancerology. It is a very popular tropical aquarium fish species (Danio rerio). The zebrafish is a vertebrate, the genome of which is quite close to that of humans [Barbazuk et al., Genome Res., 10(9): 1351-1358, 2000] [20], hence its advantage as an animal model applied to human pathological conditions. Its very fast growth time, with an organism which reaches the adult stage in 3 days, constitutes a second advantage. The eggs and the embryo are transparent, facilitating its microscopic examination. The females lay 100 to 200 eggs per week, which facilitates statistical analyses. Furthermore, the zebrafish is very easy and inexpensive to rear [Spence et al., Biol. Rev. Camb. Philos. Soc., 83(1): 13-34, 2008] [21]. This fish develops pathological conditions close to those described in humans, and in particular: spontaneous tumours [Spitsbergen et al., Toxicol. Pathol., 28(5): 705-715, 2000; Spitsbergen et al., Toxicol. Pathol., 28(5): 716-725, 2000; Beckwith et al., Lab. Invest., 80(3): 379-385, 2000] [22, 23, 24]; cancers due to a mutation of a tumour suppressor gene [Maclnnes et al., Proc. Natl. Acad. Sci. USA, 105(30): 10408-10413, 2008] [25]. This model also offers the possibility of performing tumour xenographs [White et al., Cell Stem Cell, 2(2): 183-189, 2008; Mizgireuv et al., Cancer Res., 66(6): 3120-3125, 2006] [26, 27] and of developing transgenic models, for example for melanoma [Patton et al., Current Biol., 15(3): 249-254, 2005] [28].

A model of xenograft in the zebrafish embryo was used to study the role of Sig1R in the invasive potential of K562 cells. Briefly, the tumour cells were first incubated with a fluorescent vital cell marker (CM-Dil) and then injected into the yolk sac of the embryos.

The invasive capacity of the cells was quantified by counting the number of cells having migrated out of the yolk sac and colonized the animal's body 48 h after the injection [Spitsbergen et al., 2000; Patton et al., 2005, mentioned above] [22, 28]. These experiments were carried out in collaboration with Dr. ML Cayuela (Murcia, Spain).

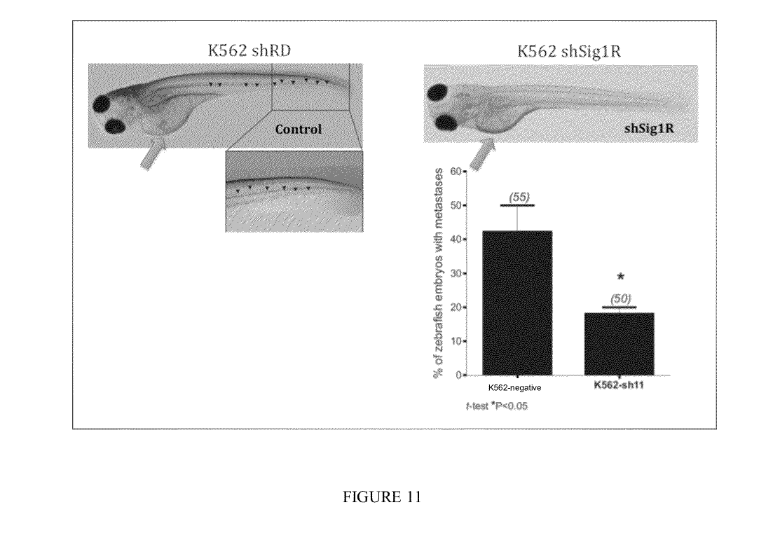

The results showed that the Sig1R silencing decreased by close to 60% the invasive capacity in vivo (FIG. 11). This makes it possible to confirm, for the first time, that Sig1R is directly involved in cancer cell migration and invasion processes.

The results make it possible to propose Sig1R as an anticancer therapeutic target.

### LIST OF REFERENCES

1. Aydar E. et al., Cancer Lett., 242: 245-257, 2006
2. Spruce B. et al., Cancer Res., 64: 4875-4886, 2004
3. Fontanilla D. et al., Science, 323: 934-937, 2009
4. Carnally S M. et al., Biophys. J., 98(7):1182-91. 2010
5. Soriani O. et al., Am. J. Physiol., 277: E73-E80, 1999a
6. Soriani O. et al., J. Pharmacol. Exp. Ther., 289: 321-328, 1999b
7. Soriani O. et al., J. Pharmacol. Exp. Ther., 286: 163-171, 1998
8. Renaudo A. et al., J. Pharmacol. Exp. Ther., 311(3): 1105-1114, 2004
9. Renaudo A. et al., J. Biol. Chem., 282: 2259-2267, 2007
10. Pillozzi S. et al., Blood, 110: 1238-1250, 2007
11. Sanguinetti MC and Jurkiewicz, NK, J. Gen. Physiol., 96(1):195-215, 1990
12. Cavarra M S. et al., J. Membr. Biol., 219(1-3):49-61, 2007
13. Roman, FJ et al., J. Pharm. Pharmacol., 42, 439-440, 1989
14. Hanner M. et al., Proc. Natl. Acad. Sci. USA., 93(15):8072-8077, 1996
15. Guasti L. et al., Mol Cell Biol. 28(16) 5043-60. 2008.
16. Phartiyal P. et al., J. Biol. Chem., 283(7):3702-3707, 2008
17. Phartiyal P. et al., J. Biol. Chem., 282(13):9874-82, 2007
18. Cherubini A. et al., Mol. Biol. Cell, 16(6):2972-2983, 2005
19. Crottes et al., J. Biol. Chem., 286(32): 27947-27958, 2011
20. Barbazuk et al., Genome Res., 10(9): 1351-1358, 2000
21. Spence et al., Biological Review of the Cambridge Philosophical Society, 83: 13-34, 2008
22. Spitsbergen et al., Toxicol. Pathol., 28(5): 705-715, 2000
23. Spitsbergen et al., Toxicol. Pathol., 28(5): 716-725, 2000
24. Beckwith et al., Lab. Invest., 80(3): 379-385, 2000
25. Maclnnes et al., Proc. Natl. Acad. Sci. USA, 105(30): 10408-10413, 2008
26. White et al., Cell Stem Cell, 2(2): 183-189, 2008
27. Mizgireuv et al., Cancer Res., 66(6): 3120-3125, 2006
28. Patton et al., Current Biol., 15(3): 249-254, 2005

1. Ligand which modulates a channel macrocomplex comprising a sigma-1 receptor and an ion channel for use as a medicament intended for the post-transcriptional regulation of the membrane expression of said ion channel.

2. Ligand which modulates a channel macrocomplex comprising a sigma-1 receptor and an ion channel for the treatment of diseases involving said ion channels, chosen from the group comprising nervous system diseases, neurodegenerative diseases, heart diseases, and cancer.

3. Modulating ligand according to claim 2, for the treatment of cancer.

4. Modulating ligand according to claim 1, where the membrane expression of said ion channel is inhibited.

5. Modulating ligand according to claim 1, where the ion channel is a potassium channel.

6. Modulating ligand according to claim 5, where the potassium channel is an ERG channel.

7. Modulating ligand according to claim 2, where the ion channel is a potassium channel.

8. Modulating ligand according to claim 3, where the ion channel is a potassium channel.

9. Modulating ligand according to claim 4, where the ion channel is a potassium channel.

10. Modulating ligand according to claim 7, where the potassium channel is an ERG channel.

11. Modulating ligand according to claim 8, where the potassium channel is an ERG channel.

12. Modulating ligand according to claim 9, where the potassium channel is an ERG channel.
